SEQUENCE LISTING (16 sequences)

SEQ ID NO: 1 - DNA, 35 bp, Artificial Sequence
  Description of Artificial Sequence Synthetic primer
gaccaaactcacagtaaaaataggtgattttggtc

SEQ ID NO: 2 - DNA, 20 bp, Artificial Sequence
  Description of Artificial Sequence Synthetic primer
cccactccatcgagatttct

SEQ ID NO: 3 - DNA, 33 bp, Artificial Sequence
  Description of Artificial Sequence Synthetic primer
gaccaaacacagtaaaaataggtgattttggtc

SEQ ID NO: 4 - DNA, 34 bp, Artificial Sequence
  Description of Artificial Sequence Synthetic primer
agaccaaacacagtaaaaataggtgattttggtc

SEQ ID NO: 5 - DNA, 23 bp, Artificial Sequence
  Description of Artificial Sequence Synthetic primer
cgatggtgggacccactccatcg

SEQ ID NO: 6 - DNA, 27 bp, Artificial Sequence
  Description of Artificial Sequence Synthetic primer
aataggtgattttggtctagctacaga

SEQ ID NO: 7 - DNA, 35 bp, Artificial Sequence; identical to SEQ ID NO: 1
  Description of Artificial Sequence Synthetic primer

SEQ ID NO: 8 - DNA, 32 bp, Artificial Sequence
  Description of Artificial Sequence Synthetic primer
gaccaaaacagtaaaaataggtgattttggtc

SEQ ID NO: 9 - DNA, 22 bp, Artificial Sequence
  Description of Artificial Sequence Synthetic primer
ctcgatggagtgggtcctcgag

SEQ ID NO: 10 - DNA, 20 bp, Artificial Sequence
  Description of Artificial Sequence Synthetic primer
aagaaatctcgatggagtgg

SEQ ID NO: 11 - DNA, 23 bp, Artificial Sequence
  Description of Artificial Sequence Synthetic primer
ggtgattttggtctagctacaaa

SEQ ID NO: 12 - DNA, 31 bp, Artificial Sequence
  Description of Artificial Sequence Synthetic primer
gtagctaaataggtgattttggtctagctac

SEQ ID NO: 13 - DNA, 32 bp, Artificial Sequence
  Description of Artificial Sequence Synthetic primer
ctagacagtaaaaataggtgattttggtctag

SEQ ID NO: 14 - DNA, 20 bp, Artificial Sequence
  Description of Artificial Sequence Synthetic primer
cgatggagtgggtcccatcg

SEQ ID NO: 15 - DNA, 103 bp, Homo sapiens
ttcttcatgaagacctcacagtaaaaataggtgattttggtctagctacagtgaaatctcgatggagtgggtcccatcagtttgaacagttgtctggatccat

SEQ ID NO: 16 - DNA, 123 bp, Homo sapiens
atattcgtccacaaaatgattctgaattagctgtatcgtcaaggcactcttgcctacgccaccagctccaactaccacaagtttatattcagtcattttcagcaggccttataataaaaataa

(19) United States
(12) Patent Application Publication
(10) Pub. No.: US 2016/0002740 A1
(43) Pub. Date: Jan. 7, 2016

(54) DETECTION OF NUCLEIC ACIDS IN URINE

(71) Applicant: Trovagene, Inc., San Diego, CA (US)

(72) Inventors: Cecile Rose Vibat, Del Mar, CA (US); Vlada Melnikova, Poway, CA (US); Charles Rodi, Del Mar, CA (US)

(21) Appl. No.: 14/831,583

(22) Filed: Aug. 20, 2015

Related U.S. Application Data
(63) Continuation-in-part of application No. PCT/US2014/030080, filed on Mar. 15, 2014.
(60) Provisional application No. 61/802,131, filed on Mar. 15, 2013.

(51) Int. Cl. C12Q 1/68

(52) U.S. Cl. CPC C12Q 1/6886; C12Q 2600/156; C12Q 2600/106

(57) ABSTRACT

Provided are methods of determining whether a subject comprises a target nucleic acid sequence that is 51-110 nucleotides in length. Also provided are methods of monitoring a condition or treatment effect in a subject.

### CROSS-REFERENCE TO RELATED APPLICATIONS

This is a continuation-in-part of International Application No. PCT/US2014/030080, with an international filing date of Mar. 15, 2014, which claims the benefit of priority from U.S. Provisional Patent Application No. 61/802,131, filed Mar. 15, 2013. The above priority applications are hereby incorporated by reference in their entirety.

### BACKGROUND OF THE INVENTION

(1) Field of the Invention

The present application generally relates to the detection of cell-free transrenal nucleic acids in urine. More specifically, the application is directed to methods of detecting transrenal nucleic acids using PCR footprints of 51-110 nucleotides.

(2) Description of the Related Art

Fragmented DNA in blood crosses the kidney barrier (called transrenal DNA or Tr-DNA) and is detectable in the urine of a subject (Botezatu et al., Clin Chem. 46: 1078-1084, 2000; Su et al., J Mol. Diagn. 6:101-107, 2004; Su et al., Ann N Y Acad. Sci. 1022: 81-89, 2004). This cell-free Tr-DNA can contain diagnostic markers, such as the case of specific, known sequences that different from bulk genomic DNA. For example, detection of tumor-specific DNA that results from characteristic mutations can be used for tumor diagnostics, detection of male Y chromosome-specific sequences in urine of a pregnant woman can be used to determine the male gender of the fetus, and detection of mutations characteristic of inherited disease can provide a tool for prenatal genetic testing (Chan and Lo, Semin Cancer Biol. 12: 489-496, 2002; Goessl, Expert Rev Mol. Diagn. 3: 431-442, 2003; Su et al., J Mol. Diagn. 6: 101-107, 2004; Wataganara and Bianchi, Ann N Y Acad Sci. 1022: 90-99, 2004; Botezatu et al., Clin Chem. 46: 1078-1084, 2000; and Ding et al., Proc Natl Acad Sci USA. 101: 10762-10767, 2004).

Nucleic acid biomarkers are often very specific in nature, such as in cases of a single nucleotide substitution, an insertion of a small number of nucleotides, a deletion of a small number of nucleotides, or a recombination event. Additionally, a biomarker may be present in low concentrations in body fluids, such as cases of a low frequency event or an early stage of pregnancy or disease.

A skilled person is aware of sensitive methods for detection of specific DNA or RNA sequences based on PCR or other amplification techniques. Cell-free DNA isolated from plasma, urine, and stool by conventional silica-based methods includes DNA fragments that are about 150 basepairs, or nucleotides (Chan et al., Cancer Res. 63: 2028-2032, 2003; Botezatu et al., Clin Chem. 46: 1078-1084, 2000; Su et al., J Mol. Diagn. 6: 101-107, 2004; and Diehl et al., Gastroenterology 135: 489-98, 2008). However, it should be recognized that DNA fragmentation is quite likely random, and so a target sequence of interest is likely to be in DNA fragments that have been essentially randomly cleaved. In a population of DNA fragments produced by random cleavage, the likelihood of a given target sequence being long enough for use as a PCR template is inversely proportional to the length of the target sequence. This has been previously illustrated (such as shown in FIG. 1 of US Patent Application Publication No. US 2010/0068711 A1, which is hereby incorporated by reference in its entirety).

DNA isolated from urine with a standard silica-based method consists of two fractions —high molecular weight DNA, which originates from shed cells, and low molecular weight (150-400 basepair) fraction of TR-DNA with a significant portion in the 150-250 bp range (Botezatu et al., Clin Chem. 46: 1078-1084, 2000; and Su et al., J Mol. Diagn. 6: 101-107, 2004). A recent technique for isolation of cell-free nucleic acids from body fluids applied to the isolation of transrenal nucleic acids has revealed the presence in urine of DNA and RNA fragments much shorter than 150 base pairs (U.S. Patent Application Publication No. 20080139801). U.S. Publication 20100068711 reports the amplification of “ultra-short” PCR targets of 20-50 base pairs to detect TR-NA sequences with sufficient specificity. That report includes the indication that an increase in amplification of amplicons of 25 and 39 base pairs, but not 65 or 88 base pairs, was observed with enrichment of DNA fragments of 50 to less than 150 base pairs. Additionally, amplification of the 65 base pair amplicon was indicated as due to DNA fragments of 150 to 200 base pairs.

Another benefit in using a shorter target sequence is the possible presence of single-strand breaks, or nicks, in cell-free DNA fragments. Because a PCR reaction requires templates in their single-stranded form, the effective length of cell-free DNA fragments in plasma and urine is shorter than fragmentation from double-stranded breaks.

Therefore, there are advantages in use of a shorter target size to detect specific sequences within fragmented DNA. But the advantages are difficult to obtain when the target size is close to, or greater than, the average fragment length.

### BRIEF SUMMARY OF THE INVENTION

The present invention is based in part on the discovery that transrenal nucleic acids (TR-NA) can be effectively detected and quantified using a primer footprint that is 51-110 nucleotides. This is surprising because such footprints are not very much smaller than much of the TR-NA itself.

Thus, provided are methods of determining whether a subject comprises a target nucleic acid sequence that is 51-110 nucleotides in length. The methods comprise
(a) obtaining a urine sample from the subject;
(b) separating transrenal nucleic acids (TR-NA) in the urine sample from nucleic acids greater than 1000 nucleotides; and
(c) analyzing the separated TR-NA for the target nucleic acid sequence of 51 to 110 nucleotides in length.

Also provided are methods of monitoring a condition or treatment effect in a subject. The methods comprise periodically analyzing a target nucleic acid according to the above-described methods. In these methods, a change in the detected transrenal nucleic acids indicates a change in the condition or treatment effect.

### BRIEF DESCRIPTION OF THE DRAWINGS

FIG. 1 shows the results from Assays 1-4, real-time PCR.

FIG. 2 shows the results from Assays 10 and 11, real-time PCR.

FIG. 3 shows the results from Assays 12-14, real-time PCR.

FIG. 4 shows the results of Assay 1, using droplet digital PCR with a LUX assay.

### DETAILED DESCRIPTION OF THE INVENTION

The disclosure relates to methods for analyzing or detecting a nucleic acid sequence that is present in a sample of urine. The nucleic acid sequence may be considered a target sequence, or target nucleic acid or target nucleic acid sequence, of interest. The nucleic acid sequence is one that is present in one or more nucleic acid molecules present in urine because it crossed the blood urine barrier (or kidney barrier or kidney filtration barrier). Thus the molecule(s) may be from a part of a subject's body outside the urinary tract. In some cases, the molecule may be from a kidney cell after release of the molecule into the bloodstream before it crosses the kidney barrier. A target sequence is thus present in a transrenal nucleic acid (TR-NA) molecule.

Because cells do not pass the kidney barrier, TR-NA is expected to be cell-free in a urine sample. Due to the presence of cells from parts of the urinary tract, and nucleic acids therefrom present in urine, TR-NA of interest is separated from cells in various embodiments of methods disclosed herein. So in many embodiments, a urine sample is separated into a fraction that contains cells and a cell-free fraction containing TR-NA. In some cases, the TR-NA may be bound to proteins or protein-containing complexes in a cell-free fraction.

The present invention is based in part on the discovery that transrenal nucleic acids (TR-NA) can be effectively detected and quantified using a primer footprint that is 51-110 nucleotides. See Examples. This reflects an unexpected discovery over U.S. Publication 20100068711, by indicating that a 20-50 bp footprint as described in that publication is not necessarily required to obtain accurate and sensitive detection of target sequences in TR-NAs.

Another basis for an unexpected effect is the possible presence of single-strand breaks (nicks) in cell-free DNA fragments. Therefore, the longer the amplicon, the greater the likelihood of a single-strand break in the template sequence that prevents successful amplification of the amplicon. Stated differently, a PCR reaction uses templates in their single-stranded form, which increases the deleterious effects of single-stranded breaks in cell-free TR-DNA. These considerations have previously led to a PCR assay design that is no longer than 50 base pairs in length.

Thus, in some embodiments, methods of determining whether a subject comprises a target nucleic acid sequence that is 51-110 nucleotides in length is provided. The methods comprise (a) obtaining a urine sample from the subject;
(b) separating transrenal nucleic acids (TR-NA) in the urine sample from nucleic acids greater than 1000 nucleotides; and
(c) analyzing the separated TR-NA for the target nucleic acid sequence of 51 to 110 nucleotides in length.

In these methods, the target nucleic acid sequence (“footprint”) can be any length between 51 and 110 nucleotides, inclusive. In some embodiments, the target nucleic acid sequence is 51-90 nucleotides (nt) in length. In other embodiments, the target nucleic acid sequence is less than 90 nt, e.g., 85, 80, 75, 70, 65, 60, 55, 51, or any size in between.

These methods include a step of separating transrenal nucleic acids (TR-NA) in the urine sample from nucleic acids greater than 1000 nucleotides, in order to eliminate larger nucleic acids that are not TR-NAs, to avoid interference of those larger nucleic acids with the assay. In some embodiments, nucleic acids greater than 900, 800, 700, 600, 500, 400, 300, 250, 200, or 150 nt, or any size in between, is separated from smaller nucleic acids, to obtain the size TR-NA pool desired.

In additional embodiments, nucleic acid degradation in the urine sample may be reduced. Reducing nucleic acid degradation may include inhibiting nuclease activity by increased pH, increased salt concentration, heat inactivation, or by treating said urine sample with a compound selected from the group consisting of: ethylenediaminetetraacetic acid, guanidine-HCl guanidine isothiocyanate, N-lauroylsarcosine, or sodium dodecylsulphate. In other embodiments, the nucleic acid is treated with a chaotropic salt. In many cases, the urine sample has been held in the bladder less than 12 hours.

The separating of TR-NA from cells in urine may optionally be performed by contacting the urine sample with a solid material, such as a resin, that adsorbs the TR-NA. In some cases, the material may be washed, rinsed, or eluted to remove cells and cell associating nucleic acids. The washing, rinsing, or eluting may also advantageously separate the TR-NA from larger nucleic acids as described herein.

In some embodiments, the separating may include substantially isolating the TR-NA of interest. In some cases, the isolation can be by precipitation or using a solid adsorbent material. In other cases, the separating may be by filtering the urine sample to remove cells and cell-associated nucleic acids and/or other contaminants. Optionally, the filtering may be of the solid material to which the TR-NA is bound. In some instances, the filtering removes cells and nucleic acids comprising more than about 1000 nucleotides.

The target nucleic acid sequence may be DNA or RNA, e.g., mRNA. If mRNA, that nucleic acid may be reverse transcribed into cDNA, by methods known in the art.

In various embodiments, e.g., when monitoring the amount of a cancer-causing gene mutation to follow the progression of the cancer, the target nucleic acid sequence is quantified.

The analyzing may encompass any molecular techniques now known or later discovered. Non-limiting examples include sequencing, hybridization, cycling probe reaction, polymerase chain reaction (PCR), digital PCR, nested PCR, PCR to analyze single strand conformation polymorphisms, ligase chain reaction, single nucleotide extension, extension-ligation-based methods, molecular beacons, scorpions, strand displacement amplification, PCR to analyze a restriction fragments length polymorphism, or another allele-specific method.

In some embodiments, the analyzing comprises (i) contacting the TR-NA with two primers under conditions where one primer hybridizes to the target nucleic acid sequence and the other primer hybridizes to the complement of the target nucleic acid sequence; (ii) amplifying the target nucleic acid sequence; and (iii) detecting the amplified target nucleic acid sequence.

Techniques for nucleic acid manipulation useful for the practice of the present invention are described in a variety of references, including but not limited to, Molecular Cloning: A Laboratory Manual, 2nd ed., Vol. 1-3, eds. Sambrook et al. Cold Spring Harbor Laboratory Press (1989); and current Protocols in Molecular Biology, eds. Ausubel et al., Greene Publishing and Wiley-Interscience: New York (1987) and periodic updates. Specific descriptions, while not intended to limit the scope of the present invention, provide guidance in practicing certain aspects of the present invention.

To assist in detection and analysis, specific DNA sequences can be “amplified” in a number of ways, including, but not limited to cycling probe reaction (Bekkaoui, F. et al, BioTechniques 20, 240-248 (1996), polymerase chain reaction (PCR), nested PCR, PCR-SSCP (single strand conformation polymorphism), ligase chain reaction (LCR) (F. Barany Proc. Natl. Acad. Sci. USA 88: 189-93 (1991)), cloning, strand displacement amplification (SDA) (G. K. Terrance Walker et al., Nucleic Acids Res. 22:2670-77 (1994), and variations such as allele-specific amplification (ASA).

An alternative to amplification of a specific DNA sequence that can be used to indicate the presence of that sequence in methods of the present invention is based on hybridization of a nucleic acid cleavage structure with the specific sequence, followed by cleavage of the cleavage structure in a site-specific manner. This method is herein referred to as “cleavage product detection.” This method is described in detail in U.S. Pat. Nos. 5,541,331 and 5,614,402, and PCT publication Nos. WO 94/29482 and WO 97/27214. It allows for the detection of small amounts of specific nucleic acid sequences without amplifying the DNA sequence of interest.

In some embodiments, primer extension is used to sequence the TR-DNA. The analysis or detection of a disclosed method may thus comprise sequencing one or more TR-DNA molecules. Any sequencing method known to the skilled person may be applied in the practice of a disclosed method comprising sequencing. Non-limiting examples include “single molecule” sequencing methods and methods based upon the detection of reaction products from the DNA polymerization reaction.

In other embodiments, a pair of primers is used, and primer extension is used to synthesize double-stranded DNA (dsDNA). In some cases, the primers are used to amplify a TR-DNA sequence, such as by a method based upon the polymerase chain reaction (PCR). The disclosure thus includes a process for synthesizing multiple copies of, or amplifying, a dsDNA molecule from a TR-DNA by extending a forward primer and a reverse primer in the presence of a template DNA molecule(s). In various embodiments, the analyzing further comprises droplet digital PCR or real-time PCR, e.g., as in the Examples.

One method of detecting and analyzing TR-DNA targets utilizes specific primers with internally labeled fluorophores. Primer pairs may be designed for a DNA target of interest such that the primer binding sites lack any intervening sequences in the double stranded PCR product. That is, the primer target sequences are immediately adjacent to each other or overlapping. The each of the primers in the primer pair are internally labeled with a fluorophore near 3′-end. Appropriate fluorophores are selected from those known in the art. In some embodiments, the fluorophores are 6-carboxyfluorescein and carboxy-X-rhodamine. In many cases, the two bases closest to the 3′ end are unlabeled to ensure unhindered initiation of the DNA polymerization reaction. The fluorophores are spaced such that 6-11 bases are between the fluorophores on the two primers. In some cases, the spacing between fluorophores is 6-10 bases. Following binding of the labeled primers and inclusion of the appropriate materials required for PCR amplification, a PCR reaction amplifies the target sequence generating double-stranded oligonucleotide products, trans-labeled with the two fluorophores in close proximity. Amplified labeled product is then detected by Forster resonance energy transfer (FRET)-dependent fluorescence. In some embodiments, non-specific products are differentiated from specific products by measuring melting (dissociation) temperature.

Another method of detecting and analyzing DNA targets utilizes specific primers comprising oligonucleotide tails at the 5′ ends of their target-binding sequences. These oligonucleotide tails are labeled at their 5′ ends with appropriate fluorophores. In some embodiments, the oligonucleotide tails have no homology to any other sequences in the reaction, except short sequences adjacent to the fluorophores that are designed to be complementary to sequences on the opposite primer pair oligonucleotide tail, such that, if the two oligonucleotide tails are brought into close proximity, they will bind to each other. Each primer in the primer pair contains a replication blocking base to separate the target-binding region from the oligonucleotide tail comprising the fluorophore. This ensures that the tails are not replicated during PCR and remain single stranded. Any replication blocking base known in the art may be utilized, such as, iso-dC. Following binding of the labeled primers and inclusion of the appropriate materials required for PCR amplification, and a PCR reaction amplifies the target sequence generating double-stranded oligonucleotide products. The complementary sequences of the oligonucleotide tails anneal, bringing the fluorophore pairs into close proximity. The amplified labeled product is then detected by FRET-dependent fluorescence. Fluorescence is measured at a temperature at which sticky ends are annealed only if they are part of the same double-stranded PCR product molecule.

Another method of detecting and analyzing DNA targets utilizes three sequence-specific components, including a TaqMan probe. This method permits for very short amplicons by means of a partial target recognition sequence overlap of the TaqMan probe with the sense (same-strand) target-specific primer. This method utilizes a two stage, single tube, qPCR scheme. In stage 1, the target DNA is which is amplified using primers P1 and P2, which map in very close proximity to each other on the target sequence, thus allowing for very short templates. Primer P1 carries a target-unrelated 5′-end extension sequence, which is incorporated into the intermediate PCR products IP1/IP2 along with the template sequence. The resulting intermediate PCR product IP2 is sufficiently long to serve as template in stage 2, which involves primers P3 and P2 and a TaqMan probe Pr, which is labeled with fluorophore and quencher. The mechanics of stage 2 are largely identical to those of a standard TaqMan qPCR assay. During this stage, as in a standard TaqMan qPCR assay, the amount of the final PCR product is monitored by measuring the increase in fluorescence of the PCR mixture. The three target-specific components in the assay are primers P3 and P2 and the TaqMan probe (Pr). Determination of the annealing temperatures (Tₐ) of the participant oligonucleotides, their concentrations, extension temperatures, and the number of cycles in each stage is an important part of assay development.

In some embodiments, one primer comprises a fluorescent dye and a portion that does not hybridize to the target nucleic acid sequence, wherein the portion forms a hairpin with a second portion such that the hairpin suppresses detectable fluorescence from the fluorescent dye. See Example 2.

These methods are not limited to any particular purpose for detecting the target nucleic acid sequence. In some embodiments, the target nucleic acid sequence is not from the subject (i.e., is non-host). Non-limiting examples include TR-NA from a transplanted tissue, from a fetus in the case of a maternal subject or patient, and a pathogen.

A pathogen is a biological agent that can cause disease to its host. A synonym of pathogen is “infectious agent”. The term “pathogen” is most often used for agents that disrupt the normal physiology of a multicellular organism. A pathogen may be selected from a virus, a bacterium, a fungus, a mycoplasma, and a protozoan.

Infection is the invasion and multiplication of microorganisms in body tissues, which may be clinically unapparent or result in local cellular injury due to competitive metabolism, toxins, intracellular replication or antigen antibody response.

The methods of the disclosure are applicable to all viral pathogenic agents, including RNA, DNA, episomal, and integrative viruses. They also apply to recombinant viruses, such as the adenoviruses or lentiviruses utilized in gene therapy. Examples of infectious virus include: Retroviridae (e.g., human immunodeficiency viruses, such as HIV-1, also referred to as HTLV-III, LAV or HTLV-III/LAV, or HIV-111; and other isolates, such as HIV-LP; Picomaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Fidoviridae (e.g., ebola viruses); Paramyxoviridae (e.g., parainfluenza viruses, mumps virus, measles virus, respiratory syncytial virus); Orthomyxoviridae (e.g., influenza viruses); Buiigaviridae (e.g., Hantaan viruses, bunga viruses, phleboviruses and Nairo viruses); Arenaviridae (hemorrhagic fever virus); Reoviridae (e.g., reoviruses, orbiviruses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herperviridae (herpes simplex virus (HSV) 1 and 2, varicella zoster virus, cytomegalovirus (CMV), herpes viruses); Poxyiridae (variola viruses, vaccinia viruses, pox viruses); and lridoviridae (e.g., African swine fever virus); and unclassified viruses (e.g., the etiological agents of Spongiform encephalopathies, the agent of delta hepatitides (thought to be a defective satellite of hepatitis B virus), the agents of non-A, non-B hepatitis (class I-internally transmitted; class 2-parenterally transmitted (i.e., Hepatitis C); Norwalk and related viruses, and astroviruses).

The methods of the invention are applicable to all bacterial pathogenic agents. Examples of infectious bacteria include: Helicobacter pyloris, Borrelia (e.g., Borrelia afzelii, Bonelia anserine, Bonelia burgdorferi, Borrelia garinii, Bonelia hermsii, Borrelia recurrentis, Borrelia valaisiana, and Borrelia vincentii); Rickettsia (e.g., Rickettsia felis, Rickettsia prowazekii, Rickettsia rickettsii, Rickettsia typhi, Rickettsia conorii, Rickettsia africae, or Rickettsia akari); Legionella pneumophilia, Mycobacteria sps (e.g., M. tuberculosis, M. avium, M. intracellulare, M. kansaii M. gordonae), Staphylococcus aureus, Neisseria gonorrhoeae, Neisseria meningitidis, Listeriamonocytogenes, Streptococcuspyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcuspneumoniae, pathogenic Campylobacter sp., Enterococcus sp., Haemophilus influenzae, Bacillus antracis, corynebacterium diphtheriae, corynebacteium sp., Erysipelothrix rhusiopathiae, Clostridium penfiingers, Clostridium tetani, Enterobacter erogenes, Klebsiellapneuomiae, Pasteurella multicoda, Bacteroides sp., Fusobacterium nucleatum, Sreptobacillusmoniliformis, Treponema pallidium, Treponemapertenue, Leptospira, and Actinomeycesisraelli.

Examples of infectious fungi include: Cryptococcus neoformans, Histoplasmacapsulatum, Coccidioides immitis, Blastomyces dermatitidis, Chlamydia trachomatis, Candidaalbicans. Other infectious organisms (i.e., protists) include: Plasmodium falciparum and Toxoplasma gondii.

In some embodiments, the non-viral pathogen can be Helicobacter pylori, Bacillus anthracis, Plasmodium species or Leishmania species.

The present disclosure also includes methods of analyzing a fragment of fetal TR-DNA that has crossed both the placental and kidney barriers by analyzing or detecting target TR-NA sequences as disclosed herein. In some embodiments, analyzing for the target sequence comprises obtaining a urine sample from a pregnant female and assaying for the presence of fetal polymeric DNA in the sample. In some cases, the presence of a particular unique fetal DNA sequence is indicative of a genetic disease. In other cases, the presence of an aneuploid amount of fetal DNA is indicative of a disease in the fetus.

The target fetal DNA sequence can be, as a non-limiting example, a sequence that is present only on the Y chromosome or other sequence that is present in the paternal genome and not present in the maternal genome. In some embodiments, the method determines the sex of a fetus. In other embodiments, target sequence(s) present in the paternal genome may be used to determine or confirm paternity.

In addition to providing answers to commonly asked questions from expectant couples, determination of fetal sex can also be very helpful if there is a risk of X chromosome-linked inherited disease, e.g. Hemophilia or Duchene Muscular Dystrophy.

In other embodiments, the target nucleic acid sequence comprises a mutation in a gene associated with cancer. Non-limiting examples of such genes include ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4(C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT promoter, APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM,3 KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKBIA, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP9OAA1, LYN, NOTCH2, PRKCI, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, or STAT3 gene as well as mutants thereof as known to the skilled person.

In some embodiments, the mutation is in a BRAF gene or a KRAS gene. See Examples. Exemplary mutations in those genes are BRAF V600E and the KRAS mutations G12A, G12C, G12D, G12R, G12S, G12V and G13D.

Cancer includes solid tumors, as well as hematologic tumors and/or malignancies. Various cancers to be treated include but are not limited to hepatocellular carcinoma (HCC), gastric cancer, stomach cancer, breast cancer, lung cancer, colorectal cancer, pancreatic cancer, ovarian cancer, prostate cancer, renal carcinoma, hepatoma, brain cancer, melanoma, multiple myeloma, lymphoma, Hodgkin's lymphoma, non-Hodgkin's lymphoma, childhood lymphomas, and lymphomas of lymphocytic and cutaneous origin, leukemia, childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia, myeloid neoplasms, mast cell neoplasms, hematologic tumor, and lymphoid tumor, including metastatic lesions in other tissues or organs distant from the primary tumor site. Cancers to be treated include but are not limited to sarcoma, carcinoma, and adenocarcinoma.

In yet additional embodiments, the disclosed methods may be applied to diagnose any of the more than 3000 genetic diseases currently known (e.g. hemophilias, thalassemias, Duchenne muscular dystrophy, Huntington's disease, Alzheimer's disease and cystic fibrosis). Any genetic disease for which the mutation(s) or other modification(s) and the surrounding nucleotide sequence is known can be identified by methods of the disclosure. Some diseases may be linked to known variations in methylation of nucleic acids that can also be identified by methods of the present invention.

Further, there is growing evidence that some DNA sequences can predispose an individual to any of a number of diseases such as diabetes, arteriosclerosis, obesity, various autoimmune diseases and cancer (e.g. colorectal, breast, ovarian, lung), or chromosomal abnormality (either prenatally or postnatally). The diagnosis for a genetic disease, chromosomal aneuploidy or genetic predisposition can be performed prenatally by collecting an appropriate bodily fluid, such as, urine from the pregnant mother. With the advent of broad-based genetic mapping initiatives such as the Human Genome Project, there is an expanding list of targets and applications for the disclosed methods. Many diseases will be easily detectable by analysis of TR-DNA, such as fetal TR-DNA from a pregnant female. These include Marfan Syndrome, Sickle Cell Anemia, Tay Sachs Disease, and a group of neurodegenerative disorders, including Huntington's Disease, Spinocerebellar Ataxia 1, Machado-Joseph Disease, Dentatorubraopallidoluysian Atrophy, and others. Urine DNA analysis can detect the presence of the mutant gene inherited from the father. Also, if the mother's genome bears a mutation, the test can help determine whether a normal version of the gene has been inherited from the father.

Also provided are methods of monitoring a condition or treatment effect in a subject. The methods comprise periodically analyzing a target nucleic acid according to the above-described methods. In these methods, a change in the detected transrenal nucleic acids indicates a change in the condition or treatment effect.

Non-limiting examples include daily or near daily intervals, weekly or near weekly intervals, monthly or near monthly intervals, bimonthly or near bimonthly intervals, semiannual or nearly semiannual intervals, annual or nearly annual intervals, or biannual or nearly biannual intervals. In some embodiments, the monitoring is to assess the rejection or acceptance of transplanted cells, tissue or organ. In other embodiments, the monitoring may be to assess the results of a treatment, such as surgical removal of cancer cells, chemotherapy, or radiation therapy as non-limiting examples.

Embodiments involving monitoring of treatment methods may be used to address a clinical challenge for tumor chemotherapy. That is the variable sensitivity of different tumors to anti-tumor drugs, and the absence of a simple test for the quick early stage evaluation of anti-tumor therapy. Normally, the oncologist can observe the results of treatment only after several months. Meanwhile, the tumor can continue to grow and possibly metastasize if the chemotherapeutic regimen is ineffective. One embodiment of the present disclosure, useful for the immediate monitoring of the effectiveness of tumor therapy, is the quantitative analysis of tumor-specific mutations in the patient's urine DNA. If the treatment is effective, then more tumor cells die, and the ratio of the mutant sequence to any normal reference sequence contained in the urine will increase. Eventually, if chemotherapy is effective the mutant tumor-specific sequence will disappear. Periodic analysis of a patient's urine DNA can be used for monitoring of possible tumor re-growth. Early indication of chemotherapeutic ineffectiveness would allow time to try other chemotherapeutics and anti-tumor treatments. This approach is similarly effective for the evaluation of the effectiveness of radiation therapy and other cancer therapies and for monitoring after surgical treatment of cancerous growths.

To facilitate understanding of the invention, a number of terms are defined below.

The terms “detect” and “analyze” in relation to a nucleic acid sequence, refer to the use of any method of observing, ascertaining or quantifying signals indicating the presence of the target TR-NA sequence in a sample or the absolute or relative quantity of that target nucleic acid sequence in a sample. Methods can be combined with nucleic acid labeling methods to provide a signal by, for example: fluorescence, radioactivity, colorimetry, gravimetry, X-ray diffraction or adsorption, magnetism, enzymatic activity and the like. The signal can then be detected and/or quantified, by methods appropriate to the type of signal, to determine the presence or absence, of the specific DNA sequence of interest.

To “quantify” in relation to a nucleic acid sequence, refers to the use of any method to study the amount of a particular nucleic acid sequence, including, without limitation, methods to determine the number of copies of a nucleic acid sequence or to determine the change in quantity of copies of the nucleic acid sequence over time, or to determine the relative concentration of a sequence when compared to another sequence.

The term “gene” refers to a DNA sequence that comprises control and coding sequences necessary for the transcription of an RNA sequence. The term “genome” refers to the complete gene complement of an organism, contained in a set of chromosomes in eukaryotes.

A “wild-type” gene or gene sequence is that which is most frequently observed in a population and is thus arbitrarily designed the “normal” or “wild-type” form of the gene. In contrast, the term “modified”, “mutant”, “anomaly” or “altered” refers to a gene, sequence or gene product which displays modifications in sequence and or functional properties (i.e., altered characteristics) when compared to the wild-type gene, sequence or gene product. For example, an altered sequence detected in the urine of a patient can display a modification that occurs in DNA sequences from tumor cells and that does not occur in the patient's normal (i.e. non-cancerous) cells. It is noted that naturally-occurring mutants can be isolated; these are identified by the fact that they have altered characteristics when compared to the wild-type gene or gene product. Without limiting the invention to the detection of any specific type of anomaly, mutations can take many forms, including addition, addition-deletion, deletion, frame-shift, missense, point, reading frame shift, reverse, transition and transversion mutations as well as microsatellite alterations.

A “disease associated genetic anomaly” refers to a gene, sequence or gene product that displays modifications in sequence when compared to the wild-type gene and that is indicative of the propensity to develop or the existence of a disease in the carrier of that anomaly. A disease associated genetic anomaly encompasses, without limitation, inherited anomalies as well as new mutations.

The term “unique fetal DNA sequence” is defined as a sequence of nucleic acids that is present in the genome of the fetus, but not in the maternal genome.

The terms “oligonucleotide” and “polynucleotide” and “polymeric” nucleic acid are interchangeable and are defined as a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide can be generated in any manner, including chemical synthesis, DNA replication, reversed transcription, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also can be said to have 5′ and 3′ ends.

When two different, non-overlapping oligonucleotides anneal to different regions of the same linear complementary nucleic acid sequence, and the 3′ end of one oligonucleotide points towards the 5′ end of the other, the former can be called the “upstream” oligonucleotide and the latter the “downstream” oligonucleotide.

The term “primer” refers to an oligonucleotide which is capable of acting as a point of initiation of synthesis when placed under conditions in which primer extension is initiated. An oligonucleotide “primer” can occur naturally, as in a purified restriction digest or be produced synthetically.

A primer is selected to be “substantially” complementary to a strand of specific sequence of the template. A primer must be sufficiently complementary to hybridize with a template strand for primer extension or elongation to occur. A primer sequence need not reflect the exact sequence of the template. For example, a non-complementary nucleotide fragment can be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template primer complex for synthesis of the extension product of the primer.

A “target” nucleic acid is a nucleic acid sequence to be evaluated by hybridization, amplification or any other means of analyzing a nucleic acid sequence, including a combination of analysis methods.

“Hybridization” methods involve the annealing of a complementary sequence to the target nucleic acid (the sequence to be analyzed). The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

Hybridization encompasses, but is not limited to, slot, dot and blot hybridization techniques.

It is important for some diagnostic applications to determine whether the hybridization represents complete or partial complementarity. For example, where it is desired to detect simply the presence or absence of pathogen DNA (such as from a virus, bacterium, fungi, mycoplasma, protozoan) it is only important that the hybridization method ensures hybridization when the relevant sequence is present; conditions can be selected where both partially complementary probes and completely complementary probes will hybridize. Other diagnostic applications, however, could require that the hybridization method distinguish between partial and complete complementarity. It may be of interest to detect genetic polymorphisms.

Methods that allow for the same level of hybridization in the case of both partial as well as complete complementarity are typically unsuited for such applications; the probe will hybridize to both the normal and variant target sequence. The present invention contemplates that for some diagnostic purposes, hybridization be combined with other techniques (such as restriction enzyme analysis). Hybridization, regardless of the method used, requires some degree of complementarity between the sequence being analyzed (the target sequence) and the fragment of DNA used to perform the test (the probe). (Of course, one can obtain binding without any complementarity but this binding is nonspecific and to be avoided.)

The complement of a nucleic acid sequence as used herein refers to an oligonucleotide which, when aligned with the nucleic acid sequence such that the 5′ end of one sequence is paired with the 3′ end of the other, is in “antiparallel association.” Specific bases not commonly found in natural nucleic acids can be included in the nucleic acids of the present invention and include, for example, inosine and 7-deazaguanine. Complementarity need not be perfect; stable duplexes can contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value can be calculated by the equation: Tm=S1.5+0.41(% G+C), when a nucleic acid is in aqueous solution at 1 M NaCl (see e.g., Anderson and Young, Quantitative Filter Hybridisation, in Nucleic Acid Hybridisation (1985)). Other references include more sophisticated computations which take structural as well as sequence characteristics into account for the calculation of Tm.

The term “probe” as used herein refers to an oligonucleotide (i.e., a sequence of nucleotides), whether occurring naturally as in a purified restriction digest or produced synthetically, which forms a duplex structure or other complex with a sequence in another nucleic acid, due to complementarity or other means of reproducible attractive interaction, of at least one sequence in the probe with a sequence in the other nucleic acid. Probes are useful in the detection, identification and isolation of particular gene sequences. It is contemplated that any probe used in the present invention will be labeled with any “reporter molecule,” so that it is detectable in any detection system, including, but not limited to, enzyme (e.g., ELISA, as well as enzyme-based histochemical assays), fluorescent, radioactive and luminescent systems. It is further contemplated that the oligonucleotide of interest (i.e., to be detected) will be labeled with a reporter molecule. It is also contemplated that both the probe and oligonucleotide of interest will be labeled. It is not intended that the present invention be limited to any particular detection system or label.

The term “label” as used herein refers to any atom or molecule which can be used to provide a detectable (preferably quantifiable) signal, and which can be attached to a nucleic acid or protein. Labels provide signals detectable by any number of methods, including, but not limited to, fluorescence, radioactivity, colorimetry, gravimetry, X-ray diffraction or absorption, magnetism, and enzymatic activity.

The term “substantially single-stranded” when used in reference to a nucleic acid target means that the target molecule exists primarily as a single strand of nucleic acid in contrast to a double-stranded target which exists as two strands of nucleic acid which are held together by inter-strand base pairing interactions.

The term “sequence variation” as used herein refers to differences in nucleic acid sequence between two nucleic acid templates. For example, a wild-type structural gene and a mutant form of this wild-type structural gene can vary in sequence by the presence of single base substitutions and/or deletions or insertions of one or more nucleotides. These two forms of the structural gene are said to vary in sequence from one another. A second mutant form of the structural gene can exit. This second mutant form is said to vary in sequence from both the wild-type gene and the first mutant form of the gene.

The terms “structure probing signature,” “hybridization signature” and “hybridization profile” are used interchangeably herein to indicate the measured level of complex formation between a target nucleic acid and a probe or set of probes, such measured levels being characteristic of the target nucleic acid when compared to levels of complex formation involving reference targets or probes.

“Nucleic acid sequence” as used herein refers to an oligonucleotide, nucleotide or polynucleotide, and fragments or portions thereof, and to DNA or RNA of genomic or synthetic origin which can be single- or double-stranded, and represent the sense or antisense strand.

A “deletion” is defined as a change in either nucleotide or amino acid sequence in which one or more nucleotides or amino acid residues, respectively, are absent.

An “insertion” or “addition” is that change in a nucleotide or amino acid sequence which has resulted in the addition of one or more nucleotides or amino acid residues, respectively, as compared to, naturally occurring sequences.

A “substitution” results from the replacement of one or more nucleotides or amino acids by different nucleotides or amino acids, respectively.

A “modification” in a nucleic acid sequence refers to any change to a nucleic acid sequence, including, but not limited to a deletion, an addition, an addition-deletion, a substitution, an insertion, a reversion, a transversion, a point mutation, a microsatellite alteration, methylation or nucleotide adduct formation.

As used herein, the terms “purified”, “decontaminated” and “sterilized” refer to the removal of contaminant(s) from a sample.

As used herein, the terms “substantially purified” and “substantially isolated” refer to nucleic acid sequences that are removed from their natural environment, isolated or separated, and are preferably 60% free, more preferably 75% free, and most preferably 90% free from other components with which they are naturally associated. An “isolated polynucleotide” is therefore a substantially purified polynucleotide. It is contemplated that to practice the methods of the present invention polynucleotides can be, but need not be substantially purified. A variety of methods for the detection of nucleic acid sequences in unpurified form are known in the art.

“Amplification” is defined as the production of additional copies of a nucleic acid sequence and is generally carried out using polymerase chain reaction or other technologies well known in the art (e.g., Dieffenbach and Dveksler, PCR Primer, a Laboratory Manual, Cold Spring Harbor Press, Plainview, N.Y. [1995]). As used herein, the term “polymerase chain reaction” (“PCR”) refers to the method of K. B. Mullis (U.S. Pat. Nos. 4,683,195 and 4,683,202, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the term “polymerase” refers to any enzyme suitable for use in the amplification of nucleic acids of interest. It is intended that the term encompass such DNA polymerases as Tag DNA polymerase obtained from Thermus aquaticus, although other polymerases, both thermostable and thermolabile are also encompassed by this definition.

With PCR, it is possible to amplify a single copy of a specific target sequence in genomic DNA to a level that can be detected by several different methodologies (e.g., staining, hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications. Amplified target sequences can be used to obtain segments of DNA (e.g., genes) for insertion into recombinant vectors.

As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

As used herein, the terms “restriction endonucleases” and “restriction enzymes” refer to bacterial enzymes, each of which cut double-stranded DNA at or near a specific nucleotide sequence.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity can be “partial,” in which only some of the nucleic acids' bases are matched according to the base pairing rules. Or, there can be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There can be partial homology or complete homology (i.e., identity). A partially complementary sequence is one that at least partially inhibits a completely complementary sequence from hybridizing to a target nucleic acid is referred to using the functional term “substantially homologous.” The inhibition of hybridization of the completely complementary sequence to the target sequence can be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that are non-specific binding is permitted; low stringency conditions require that the binding of two sequences to one another be a specific (i.e., selective) interaction. The absence of non-specific binding can be tested by the use of a second target which lacks even a partial degree of complementarity (e.g., less than about 30% identity); in the absence of non-specific binding the probe will not hybridize to the second non-complementary target.

Numerous equivalent conditions can be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution can be varied to generate conditions of either low or high stringency hybridization different from, but equivalent to, the above listed conditions. The term “hybridization” as used herein includes “any process by which a strand of nucleic acid joins with a complementary strand through base pairing” (Coombs, Dictionary of Biotechnology, Stockton Press, New York N.Y. [1994].

“Stringency” typically occurs in a range from about Tm-5.degree. C. (5.degree. C. below the Tm of the probe) to about 20.degree. C. to 25.degree. C. below Tm. As will be understood by those of skill in the art, a stringent hybridization can be used to identify or detect identical polynucleotide sequences or to identify or detect similar or related polynucleotide sequences.

As used herein the term “hybridization complex” refers to a complex formed between two nucleic acid sequences by virtue of the formation of hydrogen bonds between complementary G and C bases and between complementary A and T bases; these hydrogen bonds can be further stabilized by base stacking interactions. The two complementary nucleic acid sequences hydrogen bond in an antiparallel configuration. A hybridization complex can be formed in solution (e.g., COt or ROt analysis) or between one nucleic acid sequence present in solution and another nucleic acid sequence immobilized to a solid support (e.g., a nylon membrane or a nitrocellulose filter as employed in Southern and Northern blotting, dot blotting or a glass slide as employed in situ hybridization, including FISH [fluorescent in situ hybridization]).

As used herein, the term “antisense” is used in reference to RNA sequences which are complementary to a specific RNA (e.g., mRNA) or DNA sequence. Antisense RNA can be produced by any method, including synthesis by splicing the gene(s) of interest in a reverse orientation to a viral promoter which permits the synthesis of a coding strand. Once introduced into a cell, this transcribed strand combines with natural mRNA produced by the cell to form duplexes. These duplexes then block either further transcription of the mRNA or its translation. In this manner, mutant phenotypes can be generated. The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “sample” as used herein is used in its broadest sense. A biological sample suspected of containing nucleic acid can comprise, but is not limited to, genomic DNA (in solution or bound to a solid support such as for Southern blot analysis), cDNA (in solution or bound to a solid support), and the like.

The term “urinary tract” as used herein refers to the organs and ducts which participate in the secretion and elimination of urine from the body.

The terms “transrenal DNA” and “transrenal nucleic acid” as used herein refer to nucleic acids that have crossed the kidney barrier. Transrenal DNA as used herein differs from miRNA. Specifically, transrenal DNA comprises randomness in the 3′ and 5′ ends, which is not present in miRNA.

Having now generally provided the disclosure, the same will be more readily understood through reference to the following examples which are provided by way of illustration, and are not intended to be limiting of the disclosure, unless specified.

### EXAMPLES

TABLE 1
Primers used in Examples 1 and 2. Underline indicates added non-template
bases for creation of hairpin with 3′ end at low temperatures; bold in-
dicates regular base used for preliminary assay testing by real-time
PCR; italic indicates position of mutation in the allele specific
primer; bold italic indicates base covalently linked fluorescein label.
*SID = SEQ ID NO

| General | | Strand/ | | |
|---|---|---|---|---|
| ID | Sequence | Direction | Oligo type | SID* |
| BRAF_1002 | GACCAAACTCACAGTAAAAATAGGTGATTTTGGTC | Sense | non-fluor | 1 |
| BRAF_1005 | CCCACTCCATCGAGATTTCT | Antisense | Return ASP | 2 |
| BRAF_1007 | GACCAAACACAGTAAAAATAGGTGATTTTGGTC | Sense | non-fluor | 3 |
| BRAF_1008 | AGACCAAACACAGTAAAAATAGGTGATTTTGGTC | Sense | non-fluor | 4 |
| BRAF_1010 | CGATGGTGGGACCCACTCCATCG | Antisense | non-fluor | 5 |
| BRAF_1012 | AATAGGTGATTTTGGTCTAGCTACAGA | Sense | Return ASP | 6 |
| BRAF_1019 | GACCAAACTCACAGTAAAAATAGGTGATTTTGG [symbol] C | Sense | Fluorescein- | 7 |
| | | | penultimate T | |
| BRAF_1021 | GACCAAAACAGTAAAAATAGGTGATTTTGGTC | Sense | non-fluor | 8 |
| BRAF_1024 | CTCGATGGAGTGGGTCCTCGAG | Antisense | non-fluor | 9 |
| BRAF_1027 | AAGAAATCTCGATGGAGTGG | Antisense | non-fluor | 10 |
| BRAF_1028 | GGTGATTTTGGTCTAGCTACAAA | Sense | non-fluor | 11 |
| BRAF_1036 | GTAGCTAAATAGGTGATTTTGGTCTAGCTAC | Sense | non-fluor | 12 |
| BRAF_1037 | CTAGACAGTAAAAATAGGTGATTTTGGTCTAG | Sense | non-fluor | 13 |
| BRAF_1038 | CGATGGAGTGGGTCCCATCG | Antisense | non-fluor | 14 |

### Example 1

### Detection of Amplicons of 51 or More Base Pairs

The wildtype sequence of human BRAF includes the following

(SEQ ID NO: 15)
TTCTTCATGAAGACCTCACAGTAAAAATAGGTGATTTTGGTCTAGCTACA
GTGAAATCTCGATGGAGTGGGTCCCATCAGTTTGAACAGTTGTCTGGATC
CAT

where the underlined is codon 600 (positions 1798-1800 in the genetic sequence), corresponding to a valine residue in the wildtype protein. Known mutants include V600E (with a 1799 T to A mutation), V600K (with a 1798-99 GT to AA double mutation), and V600D (with a 1799-1800 TG to AT double mutation).

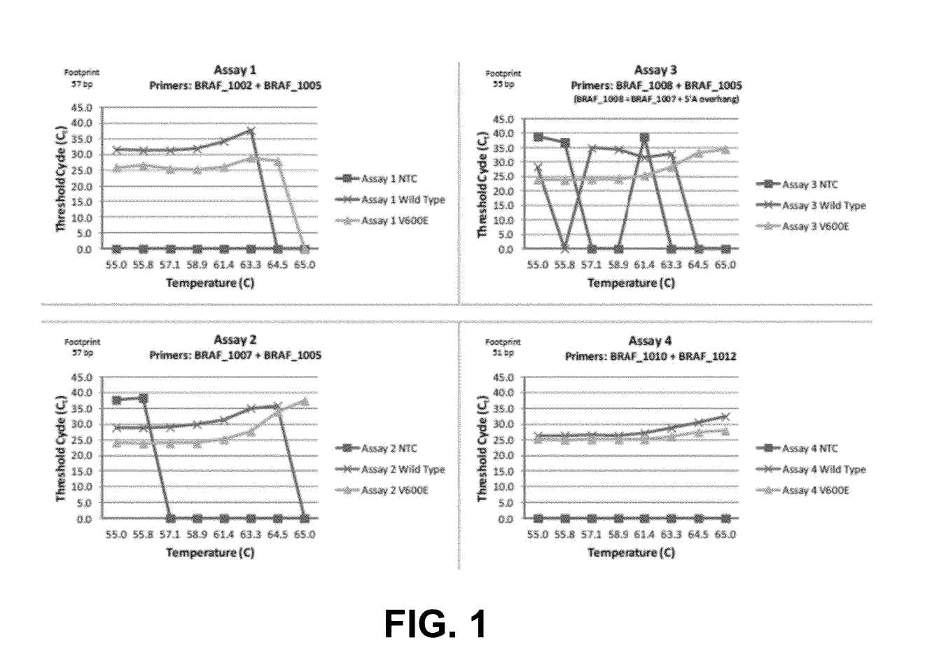

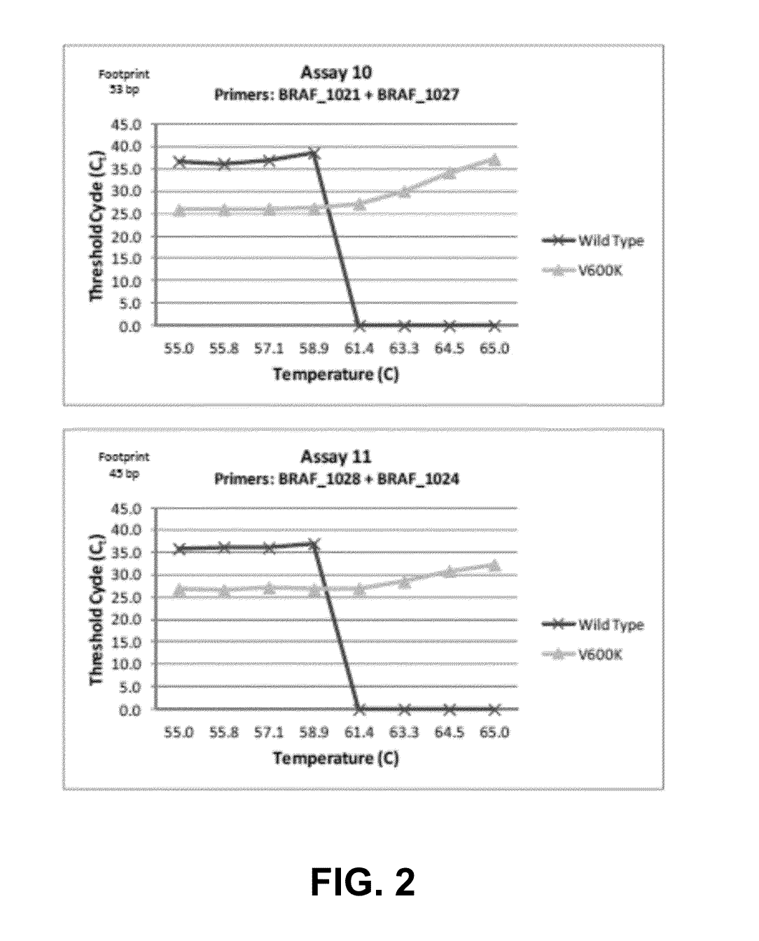

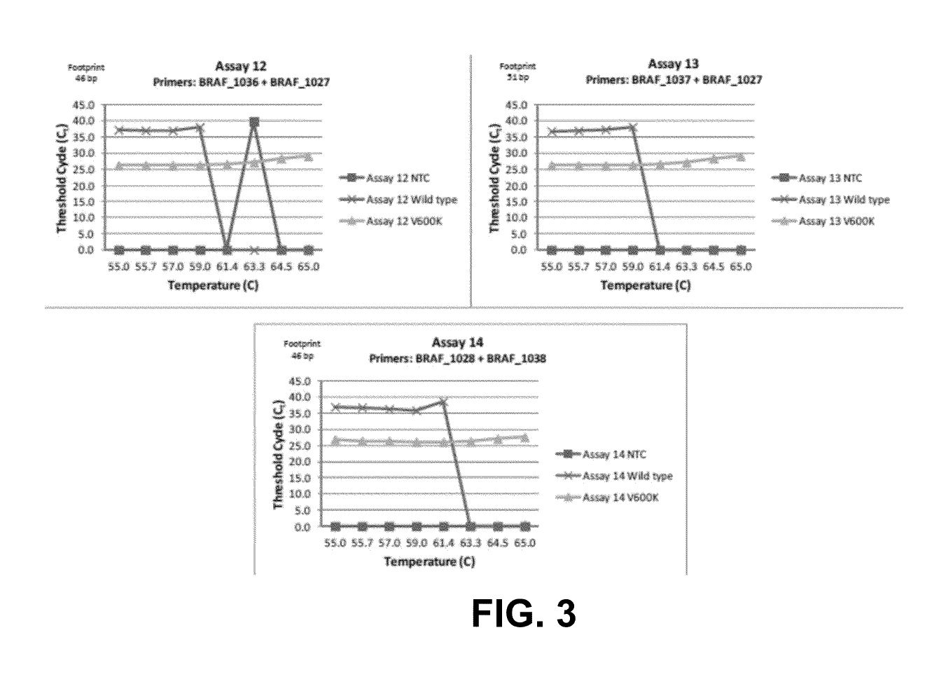

Table 2 shows the assay number, the forward and reverse primers from Table 1 for detecting V600E and V600K mutants, and the number of base pairs in the amplicon on the right. These assays use the primers indicated to generate real-time PCR data (FIGS. 1-3) to screen primer sets for their ability to differentiate between wild-type and mutant DNA sequences.

TABLE 2
Assay Characteristics

| Assay # | Forward | Reverse | BP |
|---|---|---|---|
| 1-Ex1 | BRAF_1002 | BRAF_1005 | 64 |
| 1-Ex2 | BRAF_1019 | BRAF_1005 | 57 |
| 2 | BRAF_1007 | BRAF_1005 | 65 |
| 3 | BRAF_1008 | BRAF_1005 | 62 |
| 4 | BRAF_1010 | BRAF_1012 | 63 |
| 10 | BRAF_1021 | BRAF_1027 | 60 |
| 11 | BRAF_1028 | BRAF_1024 | 52 |
| 12 | BRAF_1036 | BRAF_1027 | 52 |
| 13 | BRAF_1037 | BRAF_1027 | 57 |
| 14 | BRAF_1028 | BRAF_1038 | 50 |

FIGS. 1-3 show the results of real-time PCR of samples containing both wildtype and mutant BRAF sequences. In the figures, “NTC” refers to “no template control”.

Assay 1 (FIG. 1) is an example of an assay that could differentiate between wild-type and mutant DNA sequences. This primer set was used to develop an assay using droplet digital PCR, described in Example 2 below.

Assay 4 (FIG. 1) is an example of a primer set that does not discriminate between wild-type and mutant. It failed the screen since wt and mutant sequences are amplified at all temperatures tested.

### Example 2

### Droplet Digital LUX PCR Assay

Assay 1 from Example 1 was developed into a LUX (light-upon-extension) PCR assay with the following characteristics:

an allele-specific primer where the last base is complementary to mutant DNA (differentiating it from wild-type);

a “return primer” with the following two properties:
(a) a 5′ non-template sequence that forms a hairpin with the 3′ end; and
(b) a 5′ fluorescein label, which fluoresces when amplification of mutant sequence is amplified. The label remains quenched by the hairpin when only wild-type is present.

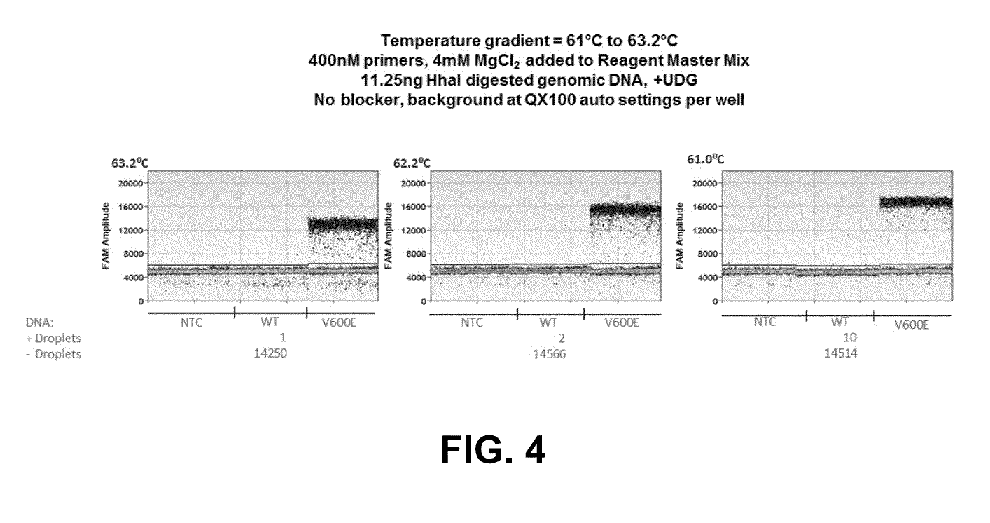

FIG. 4 shows the results of a droplet digital PCR assay using the above primers, under different temperature regimes, using genomic DNA isolated from cell lines bearing the mutant or wild-type sequence. The assay clearly differentiated the mutant from the wild-type sequence since the mutant sequence was amplified under conditions where the wild-type was not.

### Example 3

### PrimePCR ddPCR KRAS Mutation Detection Assay

Bio-Rad's KRAS G12D assay, G12D FAM, was utilized to establish the utility of detecting a 90 bp footprint in urine samples from a colorectal cancer patient. A urine specimen was obtained from a patients diagnosed with colorectal cancer who was tissue positive for the KRAS G12D mutation. Urine DNA was extracted by methods similar to those previously described in, e.g., US Patent RE39920; US Patent Pub. 2008/0139801.

Droplet digital PCR analysis was performed as follows: After combining Master Mix, DNA sample, and primer/probe mix, each sample was partitioned into approximately 20,000 nanoliter-sized droplets. Droplets were transferred to a 96-well PCR plate and PCR was performed to end point in a thermal cycler. The droplets were then streamed past a two-color optical detection system (droplet reader). Using a proprietary algorithm, the concentration of mutant and WT target were quantified.

The assay footprint is approximately 90 bp, contained within the following 123 bp sequence (the exact footprint is not known (proprietary to BioRad):

(SEQ ID NO: 16)
ATATTCGTCCACAAAATGATTCTGAATTAGCTGTATCGTCAAGGCACTCT
TGCCTACGCCACCAGCTCCAACTACCACAAGTTTATATTCAGTCATTTTC
AGCAGGCCTTATAATAAAAATAA

Results are shown in Table 3. Both replications of the assay accurately identified the KRAS G12D in the sample. This demonstrates that a 90 bp footprint is small enough to identify cancer mutations in TR-NA.

TABLE 3
Quantitation of KRAS G12D in urine using a 90 bp footprint.

| | copies/ng | copies/100k |
|---|---|---|
| Sample | input | genome equivalents |
| Patient 1 rep 1 | 7.4 | 2431 |
| Patient 1 rep 2 | 6.8 | 2246 |
| Healthy Control | 0.0 | 0 |

All references cited herein, including patents, patent applications, and publications, are hereby incorporated by reference in their entireties, whether previously specifically incorporated or not. The citation of documents herein is not to be construed as reflecting an admission that any is relevant prior art. Moreover, their citation is not an indication of a search for relevant disclosures. All statements regarding the date(s) or contents of the documents is based on available information and is not an admission as to their accuracy or correctness.

Having now fully described the inventive subject matter, it will be appreciated by those skilled in the art that the same can be performed within a wide range of equivalent parameters, concentrations, and conditions without departing from the spirit and scope of the disclosure and without undue experimentation.

While this disclosure has been described in connection with specific embodiments thereof, it will be understood that it is capable of further modifications. This application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth.

What is claimed is:

1. A method of determining whether a subject comprises a target nucleic acid sequence that is 51-110 nucleotides in length, the method comprising:
(a) obtaining a urine sample from the subject;
(b) separating transrenal nucleic acids (TR-NA) in the urine sample from nucleic acids greater than 1000 nucleotides; and
(c) analyzing the separated TR-NA for the target nucleic acid sequence of 51 to 110 nucleotides in length.

2. The method of claim 1, wherein said analyzing comprises (i) contacting the TR-NA with two primers under conditions where one primer hybridizes to the target nucleic acid sequence and the other primer hybridizes to the complement of the target nucleic acid sequence; (ii) amplifying the target nucleic acid sequence; and (iii) detecting the amplified target nucleic acid sequence.

3. The method of claim 1, wherein the target nucleic acid sequence is 51-90 nucleotides in length.

4. The method of claim 1, wherein the TR-NA is separated from nucleic acids greater than 400 base pairs.

5. The method of claim 1, wherein the TR-NA is separated from nucleic acids greater than 300 base pairs.

6. The method of claim 1, wherein the target nucleic acid sequence is quantified.

7. The method of claim 1, wherein the target nucleic acid sequence is in DNA.

8. The method of claim 1, wherein the target nucleic acid sequence is in mRNA which is reverse transcribed to cDNA.

9. The method of claim 1, wherein the analyzing comprises sequencing, hybridization, cycling probe reaction, polymerase chain reaction (PCR), digital PCR, nested PCR, PCR to analyze single strand conformation polymorphisms, ligase chain reaction, strand displacement amplification or PCR to analyze a restriction fragments length polymorphism.

10. The method of claim 2, wherein the analyzing further comprises droplet digital PCR or real-time PCR.

11. The method of claim 2, wherein one primer comprises a fluorescent dye and a portion that does not hybridize to the target nucleic acid sequence, wherein the portion forms a hairpin with a second portion such that the hairpin suppresses detectable fluorescence from the fluorescent dye.

12. The method of claim 1, wherein the target nucleic acid sequence comprises a mutation in a gene associated with cancer.

13. The method of claim 12, wherein the target nucleic acid sequence is a part of a KRAS gene or a BRAF gene.

14. The method of claim 13, wherein the mutation is a BRAF codon 600 or a KRAS codon 12 or 13 mutation.

15. The method of claim 1, wherein the target nucleic acid sequence is not from the subject.

16. The method of claim 16, wherein the target nucleic acid sequence is to a pathogen or a fetus.

17. A method of monitoring a condition or treatment effect in a subject, the method comprising
periodically analyzing a target nucleic acid according to claim 1,
wherein a change in the detected transrenal nucleic acids indicates a change in the condition or treatment effect.

18. The method of claim 17, wherein the target nucleic acid sequence is 51-90 nucleotides in length.

19. The method of claim 17, wherein the target nucleic acid sequence comprises a mutation in a gene associated with cancer.

20. The method of claim 19, wherein the target nucleic acid sequence is a part of a KRAS gene or a BRAF gene.
